(19) United States
(12) Patent Application Publication
(10) Pub. No.: US 2025/0044275 A1
(43) Pub. Date: Feb. 6, 2025

(54) EMBEDDED ELECTRODE ARRAY PLATE

(71) Applicant: AXOSIM, INC., New Orleans, LA (US)

(72) Inventors: J. Lowry Curley, New Orleans, LA (US); Corey Rountree, New Orleans, LA (US)

(21) Appl. No.: 18/717,335

(22) PCT Filed: Dec. 7, 2022
(86) PCT No.: PCT/US2022/081088

§ 371 Date: Jun. 6, 2024

Related U.S. Application Data
(60) Provisional application No. 63/286,959, filed on Dec. 7, 2021.

(51) Int. Cl. G01N 33/483; G01N 33/50; G01N 33/543

(52) U.S. Cl. CPC G01N 33/4836; G01N 33/5005; G01N 33/5438

(57) ABSTRACT

An apparatus providing an embedded electrode array (EEA) capable of testing cell samples includes an embedded electrode array, an interface printed circuit board, a test controller for configuring the stimulating signals, and data channels in the interface printed circuit board for receiving and sampling responsive signals into data channels, and a data collector for receiving data sets for processing and storage. The embedded electrode array includes tissue culture wells each having electrode pads for containing a tissue cell sample signal connectors having a connector pins for receiving a stimulating signal for each of tissue culture wells and for generating a plurality of responsive signals from each of the tissue cell samples, and circuit runs embedded within an EEA-printed circuit board, for connecting the one of the connector pins to of the electrode pads. The interface printed circuit board includes signal connectors for receiving a signal from a source for use as the stimulating signals, and amplifier-digitizer circuits.

All patents, patent applications and publications cited herein are hereby incorporated by reference in their entirety. The disclosures of these publications in their entireties are hereby incorporated by reference into this application to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### CROSS-REFERENCE TO RELATED APPLICATION

This application claims the benefit of priority to United States Provisional Patent Application No. 63/286,959, titled “EMBEDDED ELECTRODE ARRAY,” and filed on Dec. 7, 2021. The entire application is incorporated herein by reference in its entirety.

### TECHNICAL FIELD

This application relates in general to an apparatus providing a biological testing device, and more specifically, to an apparatus providing an embedded electrode array (EEA) capable of testing cell samples.

### BACKGROUND

Effects of the application of pharmacological agents upon activity within various cell types are part of most research efforts. One such cell activity tested includes peripheral nerve toxicity of the introduction of drugs to samples of representative nerve cells. This testing involves growing a cell sample in a test environment, connecting electrodes to various locations within the cell sample, electrically stimulating the cells, and recording a response observed at a sensing electrode.

Current testing procedures utilize obtaining a single electrode recording of an observed response to one or more electrical stimuli. The stimulating electrode and the sensing electrode are manually inserted into a cell sample and the one or more electrical stimuli are applied to the cell sample while the response is recorded at the sensing electrode. The stimulating electrode and the sensing electrode are electrically coupled to electronics that generate the one or more electrical stimulus and to recording electronics that digitally sample the observed response at the sensing signal. The recorded digital sample data may be stored within a computing system for later analysis.

The manual insertion of electrodes into cell samples and performing the cell stimulation and response recording has many deficiencies. Each single recording is made by hand by a technician. The technician must be trained to perform the electrode insertion into repeatable locations within a cell sample which may be challenging to train and perform. The performed steps in the testing process do not give rise to rapid, repeatable, and automated testing that may introduce variations in the testing results that ultimately impact the results obtained from a particular test. This testing procedure also prevents the repetition of testing using a set of samples for quality assurance and detecting longer-term effects on the cell sample from the application of a pharmacological agent.

Therefore, a need exists for an apparatus providing an embedded electrode array capable of testing cell samples. The present invention attempts to address the limitations and deficiencies in prior solutions according to the principles and example embodiments disclosed herein.

### SUMMARY

In accordance with the present invention, the above and other problems are solved by providing an apparatus with an embedded electrode array (EEA) capable of testing cell samples according to the principles and example embodiments disclosed herein.

In one embodiment, the present invention is an apparatus providing an embedded electrode array capable of testing cell samples. The apparatus includes an embedded electrode array, an interface printed circuit board, a test controller for configuring the one or more stimulating signals, and the plurality of data channels in the interface printed circuit board for receiving and sampling responsive signals into the plurality of data channels, and a data collector for receiving data sets for a plurality of data channels for processing and storage. The embedded electrode array includes a plurality of tissue culture wells each having a plurality of electrode pads for containing a tissue cell sample, one or more signal connectors having a plurality of connector pins for receiving a stimulating signal for each of the plurality of tissue culture wells and generating a plurality of responsive signals from each of the tissue cell samples, and a plurality of circuit runs embedded within an EEA-printed circuit board, wherein each of the plurality of circuit runs connect one of the plurality of connector pins to an electrode pad within each tissue culture well. The interface printed circuit board includes one or more signal connectors for receiving a signal from an external function generator for use as the one or more stimulating signals and a plurality of amplifier-digitizer circuits. Each of the amplifier-digitizer circuits is configured to receive one or more of the plurality of responsive signals organized into a plurality of data channels and to digitize the responsive signals for transmission as a data set for each of the plurality of data channels.

In one aspect of the present invention, each of the plurality of tissue culture wells includes a cell channel layer of culturing cells into the tissue cell sample, a pair of tissue culture well positioned at each of two ends of cell channel layer, 8 electrode pads of the plurality of electrode pads positioned across the cell channel layer, and a single electrode pad of the plurality of electrode pads within each of the pair of tissue culture wells.

In another aspect of the present invention, the embedded electrode array further includes a pair of embedded copper layers within the EEA-printed circuit board for defining the plurality of circuit runs, a base polyimide layer between the pair of embedded copper layers, a top polyimide layer on top of one of the two copper layers, and a bottom polyimide layer on top of one of the two copper layers.

In another aspect of the present invention, the embedded electrode array further in a soft-gold electroplated layer between one of the two copper layers and the top polyimide layer.

In another aspect of the present invention, each of the tissue culture wells are within the top polyimide layer exposing the plurality of electrodes within the cell channel layer and the tissue culture well.

In another aspect of the present invention, the test signal is received from an external function generator.

In another aspect of the present invention, the data sets for each of the plurality of data channels are generated by the plurality of amplifier-digitizer circuits in groups of data sets corresponding to the data set from a portion of the tissue culture wells.

In another aspect of the present invention, each the plurality of amplifier-digitizer circuits receives 16 of the one or more of responsive signals to generate 16 data channels.

In another aspect of the present invention, the interface printed circuit board comprises 16 amplifier-digitizer circuits to generated 256 data channels organized into 2 groups of 128 data channels.

In another embodiment, the present invention is an embedded electrode array that includes a plurality of tissue culture wells each having a plurality of electrode pads for containing a tissue cell sample, one or more signal connectors having a plurality of connector pins for receiving a stimulating signal for each of the plurality of tissue culture wells and generating a plurality of responsive signals from each of the tissue cell samples, and a plurality of circuit runs embedded within an EEA printed circuit board, wherein each of the plurality of circuit runs connecting one of the plurality of connector pins to an electrode pad within each tissue culture well.

In another embodiment, the present invention is an interface printed circuit board including one or more signal connectors for receiving a test signal for use as the one or more stimulating signals to a plurality of tissue culture wells in an attached embedded electrode array, one or more interface signal connectors having a plurality of connector pins for transmitting a stimulating signal for each of the plurality of tissue culture wells in the attached embedded electrode array and receiving a plurality of responsive signals from each of the tissue cell samples, and a plurality of amplifier-digitizer circuits, each configured to receive one or more of responsive signals organized into a plurality of data channels and to digitize the responsive signals for transmission as a data set for each of the plurality of data channels.

The foregoing has outlined rather broadly the features and technical advantages of the present invention so that the detailed description of the invention that follows may be better understood. Additional features and advantages of the invention will be described hereinafter that form the subject of the claims of the invention.

It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The new features that are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

Referring now to the drawings in which like reference numbers represent corresponding parts throughout:

FIG. 1 illustrates an example embodiment of cell sample testing using a manual recording aspect capable of testing cell samples according to the present invention.

FIG. 2 illustrates an apparatus providing an embedded electrode array capable of testing cell samples according to the present invention.

FIG. 3 illustrates an assembled tissue culture plate on top of the printed circuit board providing a plurality of embedded electrode arrays capable of testing cell samples according to the present invention.

FIG. 4a-c illustrate a close-up view of a connector to an embedded electrode array capable of testing cell samples according to the present invention.

FIG. 5 illustrates a printed circuit board having signal runs coupling an embedded electrode array capable of testing cell samples to a plurality of connectors according to the present invention.

FIG. 6 illustrates an apparatus providing an embedded electrode array capable of testing cell samples according to the present invention.

FIG. 7 illustrates a printed circuit board providing a plurality of embedded electrode arrays capable of testing cell samples according to the present invention.

FIG. 8 illustrates a close-up view of an embedded electrode array capable of testing cell samples according to the present invention.

FIG. 9 illustrates a printed circuit board having signal runs coupling an embedded electrode array capable of testing cell samples to a plurality of connectors according to the present invention.

FIGS. 10a-b illustrate components within an apparatus providing an embedded electrode array capable of testing cell samples according to the present invention.

FIG. 11 illustrates an example graphical representation of digital channel data collected from a plurality of tissue culture wells within an embedded electrode array 100 according to the present invention.

FIG. 12 illustrates an example graphical representation of an automated testing process to collect digital channel data from a plurality of circuit runs of an embedded electrode array 100 testing cell samples according to the present invention.

FIGS. 13a-b illustrate example results of processed sets of digital channel data collected from a printed circuit board having signal runs coupling an embedded electrode array capable of testing cell samples according to the present invention.

FIG. 14 illustrates a computer system adapted according to certain embodiments of the test controller according to the present invention.

FIG. 15 illustrates a computing system of software components of an embedded electrode array controller capable of testing cell samples according to the present invention.

### DETAILED DESCRIPTION

This application relates in general to an apparatus for a biological testing device, and more specifically, to an apparatus providing an embedded electrode array (EEA) capable of testing cell samples according to the present invention.

Various embodiments of the present invention will be described in detail with reference to the above drawings appended hereto, wherein like reference numerals represent like parts and assemblies throughout the several views. It is to be noted, however, that the drawings illustrate only selected embodiments and elements of the apparatus described herein and are therefore not to be considered limiting in scope for the apparatus as described herein and may admit to other equally effective embodiments and applications. The scope of the present invention is limited only by the scope of the claims attached hereto. Additionally, any examples set forth in this specification are not intended to be limiting and merely set forth some of the many possible embodiments for the claimed invention.

In describing embodiments of the present invention, the following terminology will be used. The singular forms “a,” “an,” and “the” include plural referents unless the context clearly dictates otherwise. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. As used herein, the singular forms “a,” “an,” and “the” are intended to include the plural forms as well, unless the context clearly indicates otherwise.

It further will be understood that the terms “comprises,” “comprising,” “includes,” and “including” specify the presence of stated features, steps, or components, but do not preclude the presence or addition of one or more other features, steps, or components. It also should be noted that in some alternative implementations, the functions and acts noted may occur out of the order noted in the figures. For example, two figures shown in succession may in fact be executed substantially concurrently or may sometimes be executed in the reverse order, depending upon the functionality and acts involved.

The terms “individual” and “user” refer to an entity, e.g., a human, using an apparatus providing an embedded electrode array capable of testing cell samples according to the present invention. The term “user” herein refers to one or more users.

The term “embedded electrode array” and “EEA-printed circuit board” refers to an apparatus having one or more culture wells for testing tissue cell samples using electrodes embedded within a printed circuit board of the array plate electrically coupling the tissue cell sample to a stimulating signal source and a signal recording circuit. The cell sample is grown within a cell channel layer in the printed circuit board extending outward from a tissue culture well. One or more electrodes are exposed to the cell sample within the cell channel layer at defined intervals that electrically couple the tissue cell sample to the stimulating signal source. The signal recording circuit is electrically coupled to the tissue cell sample within the tissue culture well. In a preferred embodiment, the cell channel layer may be constructed from a Cirlex material to create a Cirlex channel. The terms term “embedded electrode array” and “EEA-printed circuit board” may be used interchangeably.

The terms “invention” or “present invention” refer to the invention being applied for via the patent application with the title “Embedded Electrode Array Plate.” The term invention may be used interchangeably with EEA plate.

The term “printed circuit board” refers to a medium used in electrical and electronic engineering to connect electronic components to one another in a controlled manner. It takes the form of a laminated sandwich structure of conductive and insulating layers: each of the conductive layers is designed with an artwork pattern of traces, planes, and other features that are similar to wires on a flat surface that are etched from one or more sheet layers of copper laminated onto and/or between sheet layers of a non-conductive substrate. Electrical components may be fixed to conductive pads on the outer layers in the shape designed to accept the component's terminals, generally by means of soldering, to both electrically connect and mechanically fasten them to it. A separate manufacturing process may add plated-through holes that allow interconnections between layers.

The term “electrode” refers to an electrical conductor used to contact a non-metallic part of a circuit, for example, a tissue cell sample.

The term “tissue cell sample” refers to nerve cells cultured within a tissue culture well and across a Cirlex channel in a tissue culture well that contacts a plurality of electrodes.

The term “tissue culture well” refers to a recessed cavity within a printed circuit board for receiving a tissue cell sample to be tested. The tissue cell sample is cultured along a Cirlex channel and extends outward for a defined distance from a signal recording electrode positioned within the tissue culture well.

The term “Cirlex channel” refers to a recessed cavity or cell channel layer within a printed circuit board for growing a tissue cell sample to be tested. The tissue cell sample extends along the Cirlex channel for a defined distance from a signal recording electrode positioned within the tissue culture well. One or more stimulating signal electrodes are positioned at defined distances from a signal-recording electrode located within the tissue culture well. The terms Circlex channel and cell channel layer may be used interchangeably.

The term “stimulating signal” refers to an electrical signal applied to a tissue cell sample via one of a plurality of electrodes in a tissue culture well.

The term “responsive signal” refers to an electrical signal observed and sampled one or more electrode in a tissue culture well in response to application of a stimulating signal to a tissue cell sample.

The term “Intan RHS amplifier chips” refers to an integrated circuit manufactured by Intan Technologies of Los Angeles, CA, providing a complete bidirectional electrophysiology interface with a plurality of independent stimulator/amplifier channels. Each channel integrates a configurable low-noise biopotential amplifier and a programmable constant-current stimulator capable of generating stimulation pulses for extracellular microelectrodes.

The term “peripheral nerve toxicity” refers to toxic neuropathy, or nerve damage, caused by exposure to toxic substances. Peripheral nerve toxicity is a form of peripheral neuropathy, damage to the nerves away from the brain and spinal cord. Peripheral neuropathy occurs in the nerves of your arms and hands or legs and feet.

The term “amplitude distribution” refers to a mathematical distribution of recorded signal values generated from a set of stimulation signals applied to tissue nerve cells at various distances across the tissue nerve cells. The mathematical distribution organizes the recorded signal values by a measured amplitude of the recorded signals at a known distance across the tissue cell sample.

The term “velocity distribution” refers to a mathematical distribution of recorded signal values generated from a set of stimulation signals applied to tissue nerve cells at various distances across the tissue nerve cells. The mathematical distribution organizes the recorded signal values by a calculated signal velocity of the recorded signals at a known distance across the tissue cell sample. Measurement of an arrival time value for a recorded signal value to be measured at a specific distance from the location of application of a stimulating signal allows the calculation of a measured velocity as the specific distance divided by the arrival time of the recorded signal.

Meanwhile, the terms or words used in the specification and the appended claims should not be construed as limited to general and dictionary meanings but, rather, interpreted based on the meanings and concepts corresponding to technical aspects of the present disclosure on the basis of the principle that the inventor is allowed to define terms appropriately for the best explanation. Accordingly, the embodiments stated herein and illustrations in the drawings are just a preferred embodiment of the present disclosure and do not fully represent the technical aspects of the present disclosure, so it should be understood that various equivalents and modifications as an alternative could be made thereto at the time the application is filed.

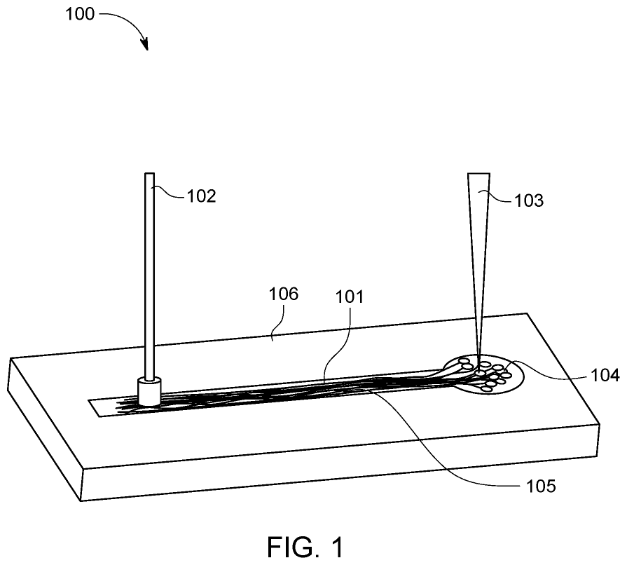

In general, the present disclosure relates to an apparatus providing an embedded electrode array (EEA) capable of testing cell samples according to the present invention. To better understand the present invention, FIG. 1 illustrates an example embodiment of cell sample testing using an apparatus providing testing cell samples according to the present invention. A cell sample testing apparatus 100 includes a tissue cell sample 101 cultured within an inner cell permissive channel 105 running within outer cell restrictive layer. The inner cell permissive channel 105 and the outer cell restrictive layer 106 may be made of either a hydrogel or a plastic like polystyrene or cyclic olefin copolymer. A recording signal electrode 103 is located within the tissue culture well 104 for recording an observed response within the tissue cell sample 101 to an applied stimulating signal. One or more stimulating electrodes 102 are located within the Cirlex channel 205 at a known distance from the recording signal electrode 103 for introducing the applied stimulating signal to the tissue cell sample 101 allowing an observed response signal within the tissue cell sample 101 by the recording signal electrode 103. The application of the stimulating signal to the tissue cell sample 101 may be repeated at each of the one or more stimulating electrodes 102 to obtain a response at a set of regular intervals along the Cirlex channel 205 to measure the transmission of the stimulating signal through the tissue cell sample 101.

While existing approaches to similar testing of tissue cell sample 101 insert the stimulating electrode 102 and recording signal electrode 103 into a tissue cell sample 101 grown within inner cell permissive channel 105, the present invention permits testing to be performed repeatedly using the stimulating electrode 102 and recording signal electrode 103 within the and the outer cell restrictive layer 106 without introducing location and distance errors from the insertion and removal of the electrodes from the tissue cell sample 101.

Examples of such testing, including the growth of a tissue cell sample 101 within inner cell permissive channel 105, are disclosed in detail within PCT Patent Application Serial No./PCT/US2015/050061, filed Sep. 14, 2015, and titled NEURAL MICROPHYSIOLOGICAL SYSTEMS AND METHODS OF USING THE SAME, and PCT Patent Application Serial No./PCT/US2018/063861/, filed Dec. 4, 2018, and titled CELL SYSTEMS USING SPHEROIDS AND METHODS OF MAKING AND USING THE SAME. These applications are commonly assigned and are incorporated by reference as if recited herein in their entireties.

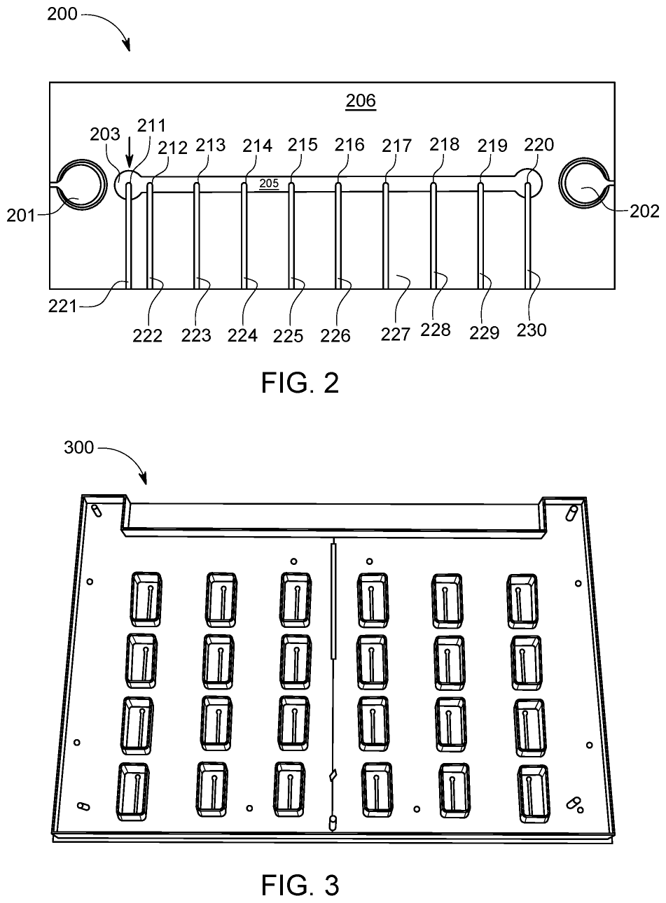

FIG. 2 illustrates an apparatus providing an embedded electrode array capable of testing cell samples according to the present invention. A single tissue culture well 200 is shown having a pair of tissue culture wells 104a-b positioned on either end of a Cirlex channel 205. A plurality of stimulating electrodes 212-219 are shown within the Cirlex channel 205 and recording signal electrodes 203, 220 are shown within each of the tissue culture well 104a-b. Each of the plurality of stimulating electrodes 212-219 exposed within the Cirlex channel 205 is located at an end of a corresponding set of circuit runs 222-229 within the PCB 206. Similarly, both of the recording signal electrodes 203, 220 are located at the end of corresponding circuit runs 221, 230.

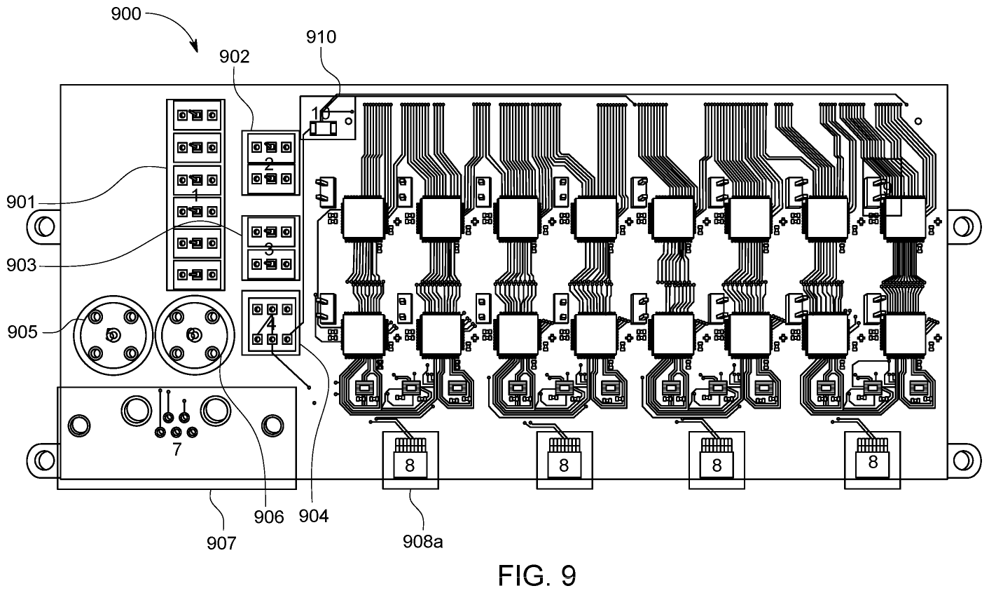

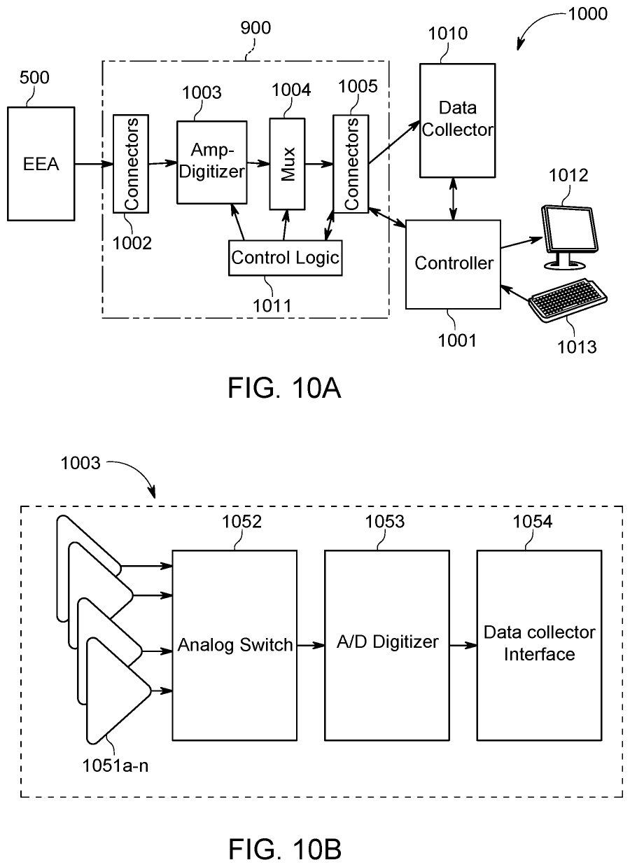

A tissue cell sample 101 is placed within one of the tissue culture wells 204a-b and cultured to extend the length of the Cirlex channel 205 while contacting each of the stimulating electrodes 212-219 and recording signal electrodes 211, 220. The set of circuit runs 221-230 is electrically coupled to a stimulating signal source and one or more signal recording circuits described below in reference to FIGS. 9-10. An automated testing system 1000 shown in FIG. 10 may be connected to the embedded electrode array 100 to automatically generate stimulating signals that are applied to the tissue cell sample 101 while responsive signals are received and acquired via the recording signal electrodes 211, 220.

A pair of reference voltage electrodes 201-202 is located adjacent to each of the tissue culture wells 204a-b. These electrodes 201-202 may apply a voltage or current here in the tissue culture well 200 to stimulate faster growth of the tissue cell sample 101 prior to testing. These electrodes 201-202 are connected to external BNC connectors (connected to the incubator and the building ground) to the ground of EEA plates to reduce noise.

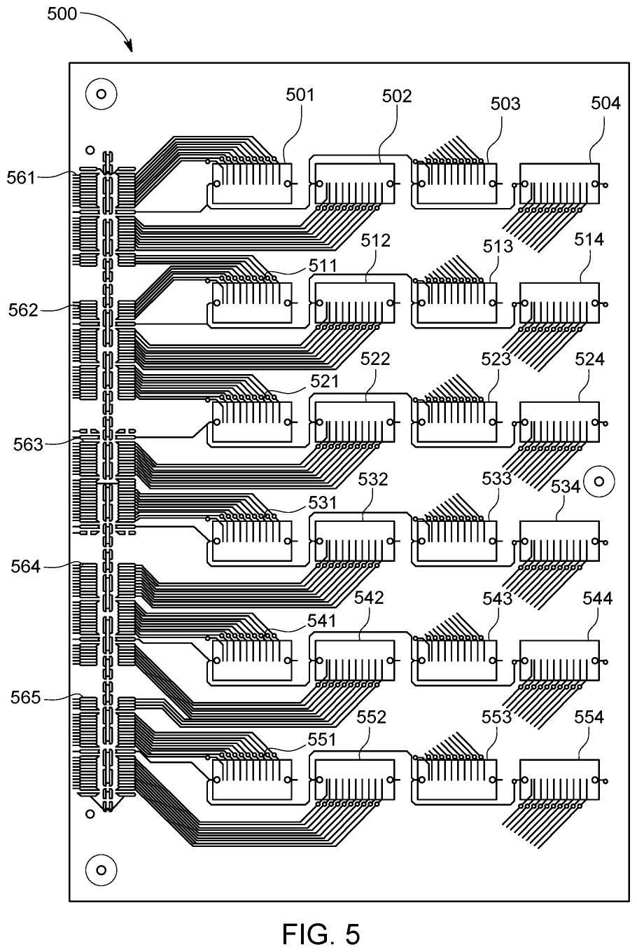

FIG. 3 illustrates a printed circuit board (PCB) providing a plurality of embedded electrode arrays capable of testing cell samples according to the present invention. The single tissue culture well 200 may be replicated a plurality of times on a single PCB 106 as shown in FIGS. 3 and 5. The set of circuit runs 222-229 is shown in FIG. 5 connecting each electrode 211-220 within the tissue culture well 200, shown in FIG. 2, to a set of connectors 561-565 attached to the PCB array 500. This arrangement of the set of circuit runs 222-229 is repeated for each of the replicated tissue culture wells 501-504, 511-514, 521-524, 531-534, 541-544, and 551-554. Each pin on the connectors 561-565 may be separately controlled within the automated testing system 1000.

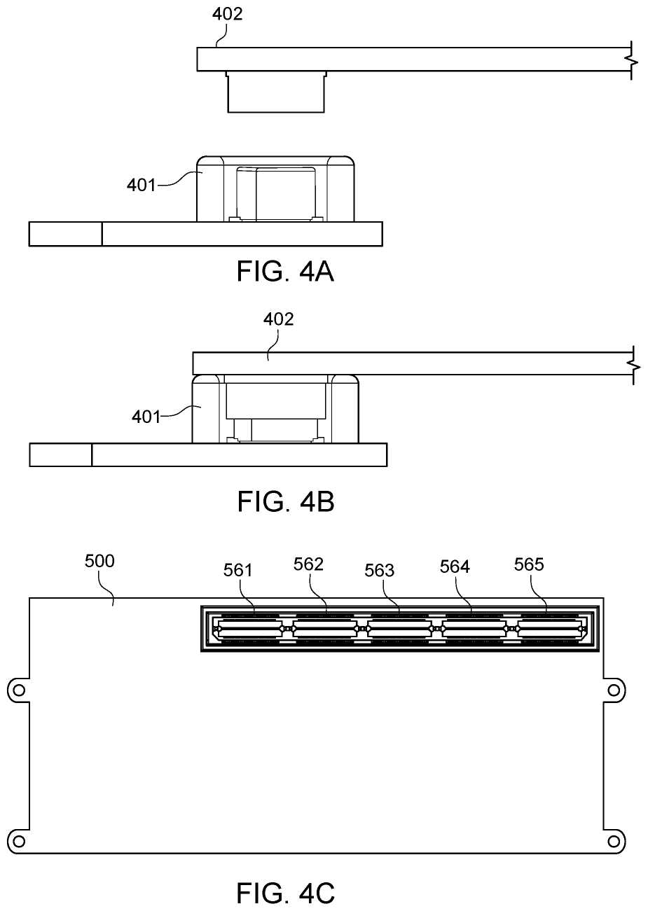

FIGS. 4a-c illustrate connecting a plurality of the embedded electrode array 100 to corresponding stimulating signal sources and the one or more signal recording circuits as shown in FIG. 9 to create the automated testing system 1000 according to the present invention. The PCB array 500 is electrically coupled to its corresponding interface PCB 900 containing the stimulating signal sources and the one or more signal recording circuits PCB board 900.

FIGS. 4a-b illustrate a coupling of PCB boards together via connectors 561-565. FIG. 4a shows the connection with a connecting cable 411 removed from its connector 412 wherein FIG. 4b shows the connecting cable 411 inserted into the connector 412. This arrangement may be repeated for each of the connectors 561-565 on the PCB array 500. Multiple connecting cables 411 may be combined into a single connector that may be attached to the connectors 561-565 as shown in FIG. 4c.

The PCB array 500 contains the plurality of tissue culture wells 200 having electrodes at the end of the set of circuit runs 221-230 passed to an interface PCB 900 shown in FIGS. 9-10 that may sample response signals from each of the recording signal electrodes 103 in each tissue culture well 200. The interface PCB 900 also includes the stimulating sources applied to the tissue cell sample 101 via the stimulating electrodes 212-219. The interface PCB 900 is controlled by a test computing system 1001 that supports programmatically defining tests to be performed on the tissue cell samples 101 in each tissue culture well 200 of the PCB array 500.

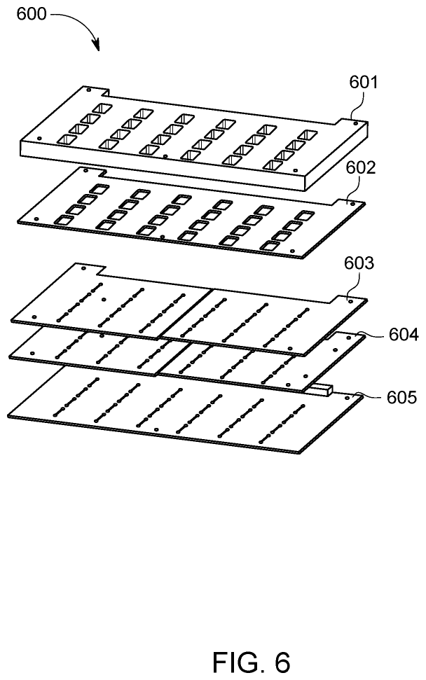

The automated testing system 1000 of FIGS. 9 and 10a-b may perform testing on a subset of the replicated tissue culture wells 501-504, 511-514, 521-524, 531-534, 541-544, and 551-554 at one time, for example, in a row of replicated tissue culture wells 501-504 . . . . Typical testing tests a top and bottom halves of the PCB array 900 separately. For instance, a test 1 would be performed on wells (501, 502, 511, 512, 521, 522, 531, 532, 541, 542, 551, 552) while a test 2 would be performed on the remainder of the wells. The set of circuit runs 222-229 is manufactured to be within the PCB array 500 as disclosed below in reference to FIGS. 6-8 and the stimulating electrodes 212-219 and recording signal electrodes 211, 220 are completed with a soft gold (Au) layer to eliminate the effects of copper on the tissue cell sample 101.

The above example describes one of a plurality of possible arrangement and uses for the electrodes 211-220. Electrodes 211-220 are all capable of stimulating and recording. A typical session will select one electrode (e.g., 215) for stimulation while recording on all of the other electrodes 211-220.

The number and arrangement of tissue culture wells 200 may be varied as needed by the requirements of any testing to be performed and may be limited by manufacturing-related considerations as to the size of the PCB array 500, the number and arrangement of connectors 561-565, and the number of channels supported by the automated testing system 1000. The set of circuit runs 222-229 on the PCB array 500 from each tissue culture well 200 may be arranged in any number of layers within the PCB array 500 as may be required by the number of supported channels.

FIG. 6 illustrates an apparatus providing an embedded electrode array capable of testing cell samples according to the present invention. An exploded view 600 of the embedded electrode array 500 includes an acrylic well layer 601, a PSA (pressure sensitive adhesive) well layer 602, a Cirlex channel layer 603, a PSA channel layer 604, and a PCB layer 605.

The acrylic well layer 601 forms separated media reservoirs for each of the plurality of tissue culture wells 200 on the PCB array 500. This acrylic well layer 601 is laser cut using a CO₂ laser to create the desired pattern. Other embodiments the embedded electrode array 500 may also include an injection molded array board made from a polycarbonate material.

The PSA well layer 602 is an adhesive that bonds the well layer 601 to the channel layer 603 and the PCB layer 605 to form media reservoirs for each tissue culture well 200. This PSA well layer 602 is an Adhesive Research pressure sensitive adhesive that is 81 μm thick. The PSA well layer 602 is laser cut using a CO₂ laser to create the desired pattern.

The Cirlex channel layer 603 forms the cell-restrictive mold that guides axonal growth across the electrodes 211-220. The Cirlex channel layer 603 is a type of polyimide called Cirlex that is nominally 500 μm thick. Cirlex is manufactured by Fralock of Valencia, CA, and may be laser cut using a UV laser to create the desired pattern of channels within the tissue culture well 200.

The PSA channel layer 604 is an adhesive that bonds the channel layer to the PCB layer 605 to form the cell-restrictive mold that guides axonal growth across the electrodes. This adhesive material is an Adhesive Research pressure sensitive adhesive that is 81 μm thick. It is laser cut using a UV laser to create the desired pattern

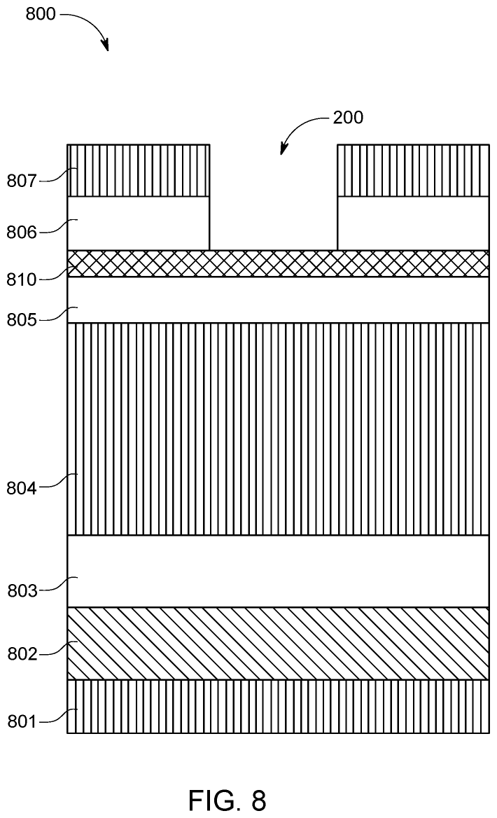

The PCB layer 605 corresponds to a flex PCB material of the PCB array 500 containing the actual embedded electrode arrays. This is a composite device consisting of multiple layers of polyimide, soft gold electroplated on copper, and adhesive layers. This PCB layer is described in detail with reference to FIG. 8 described below.

Each of the above layers 601-605 is separately manufactured with the completed layers joined into a single embedded electrode array 100. Once the layers are joined into a single device, the connectors 561-565 may be inserted and soldered in place before the entire embedded electrode array 100 is connected to the automated testing system 1000.

The plurality of tissue culture wells 200 of the PCB array 500 are created by cutting openings, cavities, and channels in each of these layers. When the above layers 601-605 are coupled together, the electrodes 211-220 are exposed within the tissue culture wells 200 when the openings, cavities, and channels in each of these layers are properly aligned. The Cirlex channel 205 and the tissue culture wells 104 in each tissue culture well 200 are also exposed as each opening above any given layer when aligned, is configured to permit access to the tissue culture well 104, Cirlex channel 105, and electrodes 211-220 for use in testing a tissue cell sample 101.

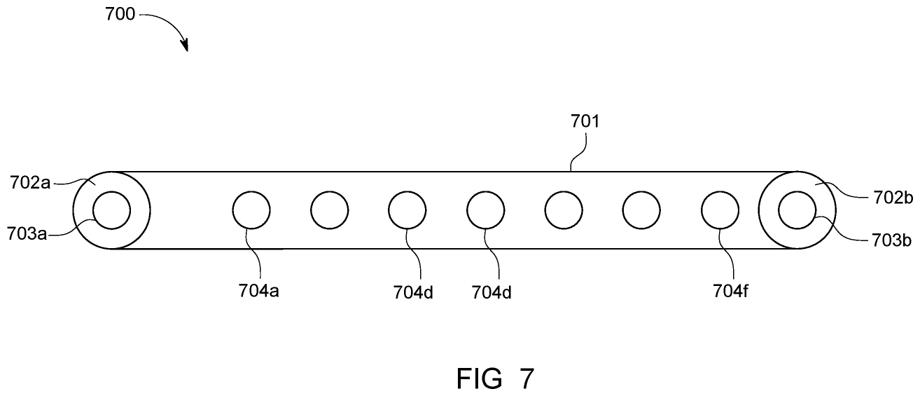

FIG. 7 illustrates a set of electrode pads within an example tissue culture well of an embedded electrode array capable of testing cell samples according to the present invention. A Cirlex channel 701 is shown having a tissue culture well 702a-b at each end. An electrode pad 703a-b, 704a-f, being an end of each circuit runs 222-229 extending upward into each tissue culture well 200, is shown positioned within the tissue culture wells 702a-b and Cirlex channel 701. Each electrode pad 704a-f within the Cirlex channel 701 is 50 μm in diameter and centered along an axis running its length. The Cirlex channel 701 itself is a tissue culture well 200 μm in width. The tissue culture wells 702a-b, having a diameter of 700 μm, each possess an electrode pad 703a-b also having a diameter of 50 μm at its center.

The distance between the tissue culture wells 701a-b is 9 mm with the distance between each adjacent pair of electrode pads 704a-f in the Cirlex channel 701 1 mm. The distance between each electrode pad 703a-b within the tissue culture wells 702a-b in the PCB array is 500 μm. All of these distances are measured from a center of each electrode pad.

The location and spacing of these electrode pads 703a-b, 704a-f are controlled during manufacture to ensure that the distances between any pair of two electrode pads are known and consistent in all tissue culture wells 200 of the embedded electrode array 100. When used in testing, a stimulating signal is applied to one of these electrode pads 704a-f while responsive signals are observed at all of the other electrode pads within the tissue culture well 200. Events occurring within a stimulating signal, for example, a voltage change from one extreme of a square wave to an alternate extreme of the square wave, may be identified within the stimulating signal. Responses observed within each of the responsive signals may occur at different points in time relative to the event occurrence in the stimulating signal depending upon the distance between the electrode introducing the stimulating signal into the tissue cell sample 101 and the electrode 704a-f corresponding to the particular responsive signal. This observed delay in the occurrence of a response in each of the responsive signals and their distances between each other may be used to calculate an observed velocity of the stimulating signal along the tissue cell sample 101. The tolerance in the location and size of the electrode pads 703a-b, 704a-f constrains the possible accuracy in any measurements in the time and velocity values. These distance, time, and velocity values and the corresponding amplitude values observed in the responsive signals may be used to calculate various metrics associated with the electrical activity within the tissue cell sample 101 both with and without a presence of a pharmacological material applied to the tissue cell sample 101 in the tissue culture well 200.

FIG. 8 illustrates a close-up view of an embedded electrode array capable of testing cell samples according to the present invention. The PCB array 500 may be constructed using a multi-layer board 800 as shown in FIG. 8. A cross-section view of a tissue culture well 200 is shown within the top layers of the multi-layer board 800. The multi-layer board 800 includes a bottom polyimide cover layer 801, a first adhesive cover layer 802, a bottom copper layer 803, a polyimide base layer 405, a top copper layer 805, a second adhesive cover layer 806, a top polyimide cover layer 807, and an a soft-gold electroplated layer 810. In an alternate embodiment, an ENIG (electroless nickel immersion gold) layer may be used.

The bottom polyimide cover layer 801 is a synthetic material layer placed on a bottom side of the PCB array 500 enclosing its internal material from an environment of the PCB array 500 while in use for testing. The polyimide material is a fabric or material made from strings of polyimide monomers having a thickness of 12.5 μm. In a preferred embodiment the polyimide material corresponds to DuPont Pyralux AP coverlay and Dupont Pyralux LF coverlay manufactured by the DuPont Corporation of Wilmington, DE. Other similar polyimide materials may also be utilized.

The first adhesive cover layer 802 couples the bottom polyimide cover layer 801 to the bottom copper layer 803. The first adhesive cover layer 802 has a thickness of 12.5 μm in a preferred embodiment. The adhesive cover layer 802 is part of the DuPont coverlay, and acts as a “proprietary B-staged modified acrylic adhesive”. The more general class would be B-staged acrylic adhesive.

The bottom copper layer 803 is a first of two copper layers into which the set of circuit runs 221-230 is defined. Each of the circuit runs 221-230 is a separate layer of copper from one of the electrodes 211-220 in the tissue culture well 200 to a connecting pin within connectors 561-565. The circuit runs 222-229 are isolated from each other along the polyimide base layer 405 and configured to reduce or eliminate electrical interference from one circuit run to another. For the location where the set of circuit runs 221-230 do not exist, between a pair of circuit runs, for example, the first adhesive cover layer 802 extends upward to the polyimide base layer 405. Each copper layer may be made of 0.5 oz of copper that is approximately 17.5 μm thick.

The polyimide base layer 405 is a synthetic material layer placed within the PCB array 500 to electrically isolate the bottom copper layer 803 and the top copper layer 805 from each other. The polyimide base layer 405 also provides rigidity and structure to the PCB array 500 to support its use and the tissue cell sample 101 grown and tested therein.

The top copper layer 805 is a second of two copper layers into which the set of circuit runs 221-230 is defined. Each of the circuit runs 221-230 is a separate layer of copper from one of the electrodes 211-220 in the tissue culture well 200 to a connecting pin within connectors 561-565. The circuit runs 222-229 are isolated from each other along the polyimide base layer 405 and configured to reduce or eliminate electrical interference from one circuit run to another. All of the circuit runs 222-229 are defined on either the bottom copper layer 803 or the top copper layer 805 that may be connected together in which the copper layers are completed through the use of vias, where additional copper is plated in a through hole connecting the two layers. The electrodes themselves are not vias however as all electrodes are located on the top copper layer. One of ordinary skill recognizes that the set of circuit runs 222-229 may be defined on more than two copper layers that are separated by a separate polyimide base layer. Similar to the bottom copper layer 803, the top copper layer 805 may be made of 0.5 oz of copper that is approximately 17.5 μm thick.

The second adhesive cover layer 806 couples the top polyimide cover layer 807 to the top copper layer 805. The second adhesive cover layer 806 has a thickness of 12.5 μm in a preferred embodiment. The adhesive comes as part of the DuPont coverlay. It is a proprietary B-staged modified acrylic adhesive. Similar adhesives that are members of a more general class would be B-staged acrylic adhesive may also be used. The top copper layer 805 extends into the tissue culture well 200 along the Cirlex channel 205 as shown in FIG. 2 and described above. For the location where the set of circuit runs 221-230 do not exist, between a pair of circuit runs, for example, the second adhesive cover layer 806 extends downward to the polyimide base layer 405.

The top polyimide cover layer 807 is a synthetic material layer placed on a top side of the PCB array 500 enclosing its internal material from an environment of the PCB array 500 while in use for testing. The polyimide material is a fabric material made from strings of polyimide monomers having a thickness of 12.5 μm similar to the bottom polyimide cover layer described above. Each tissue culture well 200 corresponds to cuts through at specific locations of each tissue culture well 200. The cut through the top polyimide cover layer 807 and the second adhesive cover layer 806 exposes electrodes 211-220 within the tissue culture well 200.

The a soft-gold electroplated layer 810 is a surface plating that is electro-plated to the top copper layer 805. The ENIG layer 810 is exposed within the tissue culture well 200 to the tissue cell sample 101 and any supporting materials creating electro-pads of the electrodes 211-220 to protect them from corrosion and other abnormalities. The ENIG layer 810 has a nominal thickness of approximately 1 μm although other thicknesses may be utilized.

FIG. 9 illustrates a printed circuit board capable of testing cell samples to a plurality of connectors according to the present invention. As noted above, the automated testing system 1000 utilizes the PCB array 500 and interface PCB 900 to perform testing operations and record data associated with observed responses to stimulation. The interface PCB 900 is electrically coupled to the PCB array 500 permitting components of the interface PCB 900 to provide stimulating signals and observe corresponding responses. The entire sequence of testing operations is performed under the control of a test controller 1001 as described below in reference to FIG. 10. Data associated with observed responses to stimulating signals are collected and stored within a data collector 1010 as described below in reference to FIGS. 10a-b.

An example layout of an interface PCB 900 contains the stimulating signal sources and one or more signal recording circuits for signals observed within the tissue cell samples 101. The interface PCB 900 includes a plurality of functional signal toggle switch isolators 901, reference electrode toggle switches 902, a function generator ground toggle switch 903, a three position toggle switch 904, a digital multiplexor control connector 905, a function generator connector 906, a power supply connector 907, a controller connector 908, a plurality of reference electrode jumper connectors 909a-n, each of which is configured to connect to a corresponding one of a plurality of amplifier/A-to-D digitizer circuits 915a-n, and a connectivity check indicator 910. Each of these toggle switches and jumper-signal connectors is used to configure the automated testing system 1000 to perform testing of a tissue cell sample 101 within the tissue culture well 200 of the PCB array 500.

The plurality of functional signal toggle switches 901 allow the selection of a reference voltage source to apply a voltage to one of the 2 reference electrodes 201-202 in the tissue culture well 200. The plurality of functional signal toggle switches 901 permits these reference voltage sources apply separate conditions to 6 groups of 4 wells each. Of course, a different number of plurality of functional signal toggle switches 901 and different groupings of tissue culture well 200 to each toggle switch may be used as needed.

The reference electrode toggle switches 902 allow the selection of a connected to common ground to be used as an amplifier reference voltage to the reference electrodes in each tissue culture well 200. The setting of the toggle switch 902 to the reference electrodes in each tissue culture well 200 eliminates noise in observed responsive signals on the electrodes 211-220.

The function generator ground toggle switches 903 select a ground signal of the function generator to a common ground in the embedded electrode array 100 or the reference electrodes 201-202 in each tissue culture well 200. In a preferred embodiment, the function generator ground toggle switches 903 are set to select the common ground when testing.

The three-position toggle switch 904 selects a source for a multiplexor control signal that selects between one of two sets of data channels for use as data sources to be received, processed, and stored for later use. The embedded electrode array 100 organizes the responsive signals received on electrodes 211-220 into data channels having two sets of data channels. A control signal received via the digital multiplexor control connector 905 selects which one of the two sets is actively being connected for storage. The three-position toggle switch 904 selects this source from a first set of data channels, a second set of data channels, and a set of data channels specified in a control signal received by the embedded electrode array 100 via the digital multiplexor control connector 905. The organization of tissue culture wells 200 into a different number of sets of data channels may be used with an encoded selection value being provided to the embedded electrode array 100 to select an active set of data channels from more than 2 sets of data channels.

The digital multiplexor control connector 905 is a BNC-type connector in a preferred embodiment that controls the selection of data channels for use as data sources to be received, processed, and stored for later use. The embedded electrode array 100 organizes the responsive signals received on electrodes 211-220 into data channels having two sets of data channels. A control signal received via the digital multiplexor control connector 905 selects which one of the two sets is actively being connected for storage. The organization of tissue culture wells 200 into a different number of sets of data channels may be used with an encoded selection value being provided to the embedded electrode array 100 to select an active set of data channels from more than 2 sets of data channels.

The function generator connector 906 is a BNC-type connector in a preferred embodiment that permits a connection of an external function generator to provide a signal applied to the tissue cell sample 101 as a stimulating signal. Characteristics of the stimulating signal, including voltage amplitude, frequency, and time between signal cycles, are defined by the external function generator. This external signal is received via the function generator connector 906 and applied to the tissue cell sample 101 using one of the recording signal electrodes 103 within each tissue culture well 200.

The power supply connector 907 is a sub-D-type connector in a preferred embodiment that receives electrical power from an external power source provided to the electronics enabling its operation. The power supply connector 907 may include connections for one or more voltage levels that may be used as power to the electronics and one or more reference voltages used within the embedded electrode array 100 as otherwise disclosed herein. In an alternate embodiment, the power supply connector 907 may accept an AC voltage that is provided to a transformer-based power supply to generate the needed power voltage and reference voltages required within the embedded electrode array 100.

The controller connector 908 is an SPI connector in a preferred embodiment that connects the embedded electrode array 100 to an external test controller 1001 disclosed in reference to FIG. 10 below.

The plurality of amplifier/A-to-D digitizer circuits 915a-n are configured into an operational state using the plurality of reference electrode jumper connectors 909a-n. Each of the plurality of reference electrode jumper connectors 909a-n is electrically coupled to a corresponding one of an amplifier/A-to-D digitizer circuits 915a-n.

The connectivity check indicator 910 correspond to a button and LED that are designed to test whether the PCB array 500 and interface PCB 900 are properly connected to each other. A user presses the button, and the system checks to make sure the ground signals are connected appropriately. When properly connected, the LED light is illuminated.

FIGS. 10a-b illustrate components within an apparatus providing an embedded electrode array capable of testing cell samples according to the present invention. FIG. 10a shows a block diagram of an automated testing system 1000 utilizing the embedded electrode array 100. FIG. 10b illustrates a block diagram of an amplifier/A-to-D? digitizer circuit 1003. The automated testing system 1000 includes the embedded electrode array 100, the interface PCB 900, a test controller 1001, and a data collector 1010. The embedded electrode array 100 is one embedded electrode array PCB 500 disclosed herein.

The interface PCB 900 includes a set of EEA connectors 1002, a plurality of amplifier/A-to-D digitizer integrated circuits (ICs) 1003, a data channel multiplexor 1004, and control logic circuit 1011 as described below in reference to FIG. 10b.

The data channel multiplexor 1004 selects one of two sets of data channels active enabling data collection from the selected data channels. The embedded electrode array 100 organizes the responsive signals received on the electrodes 211-220 into data channels having two sets of data channels. A control signal received via the digital multiplexor control connector 905 provided to the data channel multiplexor 1004 selects which one of the two sets is actively being connected for storage. The organization of tissue culture wells 200 into a different number of sets of data channels may be used with an encoded selection value being provided to the embedded electrode array 100 to select an active set of data channels from more than 2 sets of data channels.

The control logic circuit 1011 receives a set of control input signals from the test controller 1001 used to generate signals controlling the various electronic components within the interface PCB 900 and the embedded electrode array 100. The control input signals may be received by the interface PCB 900 using the various connectors described above in reference to FIG. 9. The control logic circuit 1011 generates signals to control the operation of amplifier/D-to-A digitizer circuit 1003 and the data channel multiplexor 1005.

The test controller 1001 is a programmable computing device that performs operations to implement an automated tissue cell sample 101 testing process. The test controller 1001 implements an automated test process by commanding the interface PCB 900 to activate the generation of stimulating signals onto one or more stimulating electrodes 102 in tissue culture wells 200 of the embedded electrode array 100 and retrieve and store digital data sets from data channels associated with the tissue culture wells 200 receiving the stimulating signals. The test controller 1001 may repetitively repeat these operations onto alternate tissue culture wells 200 using alternate stimulating signals.

The tissue cell samples 101 must be cultivated within the Cirlex channel 205 of each tissue culture well 200 to be used in the automated testing process. The embedded electrode array 100 may then be configured to operate as otherwise disclosed here with the embedded electrode array 100 in the tissue culture wells 200 being stored within an appropriate environment before the automated testing process is initiated. The test controller 1001 may repeat the above sequence of operations until responsive signal data from each tissue cell sample 101 is collected using one of the available stimulating signals. The automated testing process may be performed using tissue cell samples 101 after required cultivation within the Cirlex channel 205 is complete to generate a baseline dataset. One or more chemical and/or pharmacological materials may be applied to one or more tissue cell samples 101 and the automated testing process may be repeated at different time periods after the application of the pharmacological material to obtain data of a time series representing the effects of the pharmacological material on the responses observed within the tissue cell samples 101. The automated testing process may use various pharmacological material applied using different amounts or concentrations of the pharmacological material in these testing processes to explore a large range of variables as part of a larger set of tests using particular pharmacological material and sets of similar pharmacological materials.

The data collector 1010 is a digital electrophysiological test device that generates stimulating signals applied to the tissue cell sample 101 within each tissue culture well 200 of the embedded electrode array 100. The data collector 1010 also collects and stores digital data sets of time-based responsive signals generated by tests performed using the embedded electrode array 100. In a preferred embodiment, the data collector 1010 may be a commercially available modular electrophysiology data acquisition system, for example, an Intan Stim/recording system from Intan Technology of Los Angeles, CA. The modular electrophysiology data acquisition system may also be provided by any equivalent commercially available systems.

FIG. 10b illustrates a block diagram of an amplifier/A-to-D digitizer circuit 1003. The interface PCB 900 includes a set of EEA connectors 1002, a plurality of amplifier/A-to-D digitizer integrated circuits (ICs) 1003, a data channel multiplexor 1004, and a control logic circuit 1011 as described below in reference to FIG. 10b.

The set of EEA connectors 1002 permits electrical signals to be passed between the interface PCB 900 and the embedded electrode array 100 using a data cable 1021. This data cable electrically couples each pin on connectors 561-565 of the embedded electrode array 100 to the pins on the set of EEA connectors 1002 to pass power voltage, reference voltage, and ground signals, and one or more stimulating signals.

The amplifier/D-to-A digitizer ICs 1003, in a block diagram form in FIG. 10b includes a plurality of signal amplifiers 1051a-n, an analog switch 1052, a digital-o-analog circuit 1053, and a data collector interface 1054. In a preferred embodiment, each of the plurality of amplifier/D-to-A digitizer ICs 1003 comprises an Intan Technologies RHS2116 digital electrophysiology stimulator/amplifier integrated circuit manufactured by Intan Technologies of Los Angeles, CA. The Intan RHS2116 processes 16 input signals received from a set of recording signal electrodes 103 in the embedded electrode array 100 to generate digital data associated with 16 separate data channels.

Each signal from one of the recording signal electrodes 103 is received and amplified by one of the signal amplifiers 1051a-n and input into one of 16 signal inputs on the analog switch 1052. The analog switch 1052 periodically selects each of the 16 signal inputs, one at a time, to pass to the digital-to-analog circuit 1053. The digital-to-analog circuit 1053 samples each of the passed input signals to generate a digital representation of the signal within a corresponding one of 16 data channels output via the data collector interface 1054.

The digital-to-analog circuit ICs 1053 is a 16-bit D/A digitizer that may also use a reference voltage in the generation of the digital representation of the input signals. The digital-to-analog circuit 1053 typically repetitively cycles through each of the 16 input signals to generate data for each of the 16 data channels within the amplifier/D-to-A digitizer circuit 1003. The amplifier/D-to-A digitizer circuit 1003 contains 16 signal inputs to generate 16 data channels, and the interface PCB 900 includes 16 amplifier/D-to-A digitizer circuit ICs 1003 enabling the embedded electrode array 100 to provide 256 data channels from the tissue culture wells 200 within the embedded electrode array 100. The number of tissue culture wells 200, amplifier/D-to-A digitizer circuit ICs 1003, and data channels of the interface PCB 900, as well as the specific organization of groups of data channels, are described herein for exemplary purposes only. The embedded electrode array 100 is not to be limited to any number of each of these items except by the limitations of the claims attached hereto.

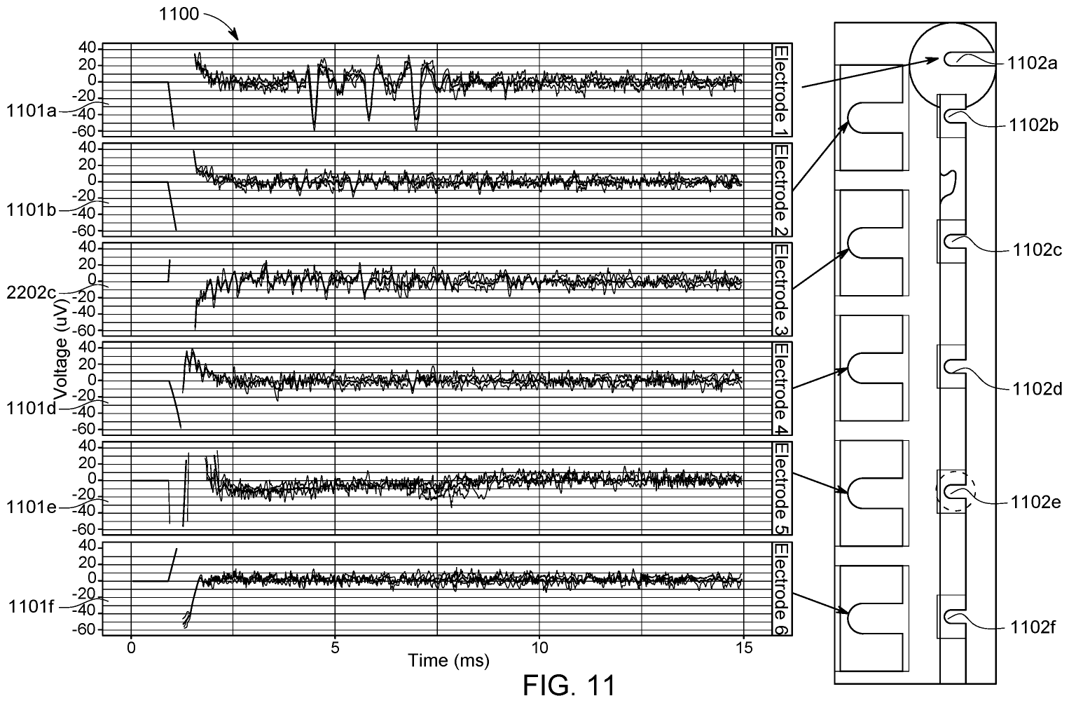

FIG. 11 illustrates an example graphical representation of digital channel data collected from a plurality of circuit runs 222-229 of an embedded electrode array 100 testing cell samples according to the present invention. FIG. 11 shows observed data sets 1101a-f from a plurality of electrodes 211-220 within one tissue culture well 200. In this example, the stimulating signal is applied to electrode 5 1102e with responsive signals observed on electrodes 1-5,6 1102a-d, f. The responsive signals are obtained within the same sampling cycle performed by an amplifier/D-to-A digitizer ICs 1003 allowing the signals to be compared. Responses seen within the various observed data sets 1101a-f may illustrate time differences in similar responses observed within the observed data sets 1101a-f as a matter of a particular electrode 1102a-f from the location of the stimulating signal.

As noted above with respect to FIG. 2, the location of each particular electrode 1102a-f and its distance relative to each other is known and consistent across all tissue culture wells 200 in the embedded electrode array 100. As such, a velocity of a response to a stimulating signal may be calculated using the time difference between a response to the stimulating signal observed on two particular electrodes 1102a-f and the known distance between these particular two electrodes 1102a-f. Additionally, a velocity may be calculated for each pair of two electrodes in a similar fashion to determine if the velocity of the observed response changes as a result of an increasing distance between the two electrodes 1102a-f.

Many different testing studies may be created using the observed data sets 1101a-f and the corresponding amplitude and location of the corresponding electrode. Because the observed data sets 1101a-f are retained within the data collector 1010, these studies may be generated using the data sets at a later date.

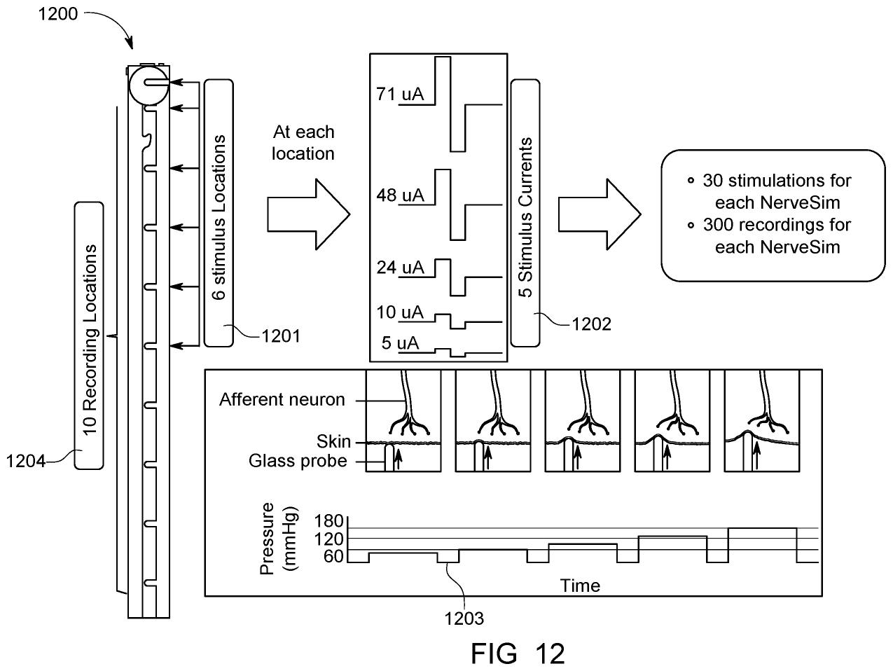

FIG. 12 illustrates an example graphical representation of an automated testing process to collect digital channel data from a plurality of circuit runs 222-229 of an embedded electrode array 100 testing cell samples according to the present invention. In this example, different stimulating signals 1202 are applied to different electrodes 1201 to observe response at one or more of the remaining electrodes within a tissue culture well 200. While multiple wells may be stimulated simultaneously, the stimulation inside each tissue culture well 200 is conducted entirely sequentially. For instance, a typical test would involve stimulating 12 tissue culture wells 200 in an upper half of the PCB array 500. The first stimuli on all 12 tissue culture wells 200 would be a low current (1 μA) stimulation at electrode 6 alone for 6 trials. The current would then be ramped up to 5 μA for 6 trials, and so forth until the max current of 64-80 μA is applied at electrode 6. The stimulus location would then shift to electrode 5 and the process would repeat. This sequence of operations is done typically for electrodes 2-6 but can be done for all 10 electrodes inside the tissue culture well 200. A pressure graphic shown in FIG. 12 is designed to explain the reasoning for increasing the stimulus current as it is similar to increasing the pressure on a touch test.

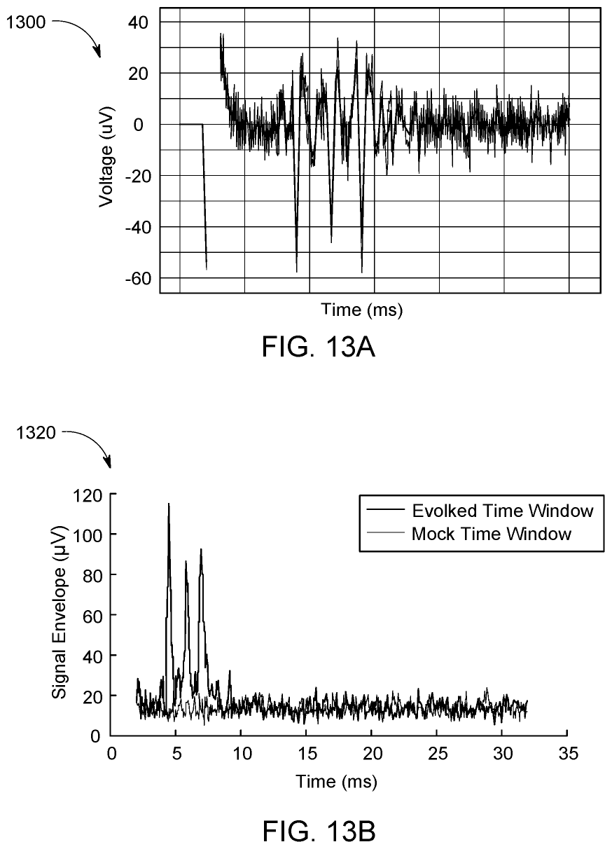

FIGS. 13a-b illustrate example results of processed sets of digital channel data collected from a printed circuit board having signal runs coupling an embedded electrode array capable of testing cell samples according to the present invention. The signals in 1300 show the processed data in the time domain while 1320 shows the same signal in the velocity domain alongside a recording of the noise levels. The velocity and amplitude distributions are obtained by performing peak finding on the signal in 1320 to find any peaks that are higher than 6 standard deviations above the noise level (red). For each peak, the velocity and amplitude, e.g., the y height shown, are extracted, and collated for the distribution. For the other metrics, the maximum velocity projection is calculated by taking a signal like 1320 from all of the responses in each well at a given current level, e.g., the 10 electrodes in well A1 were stimulated at 5 locations at 48 μA, giving 45 unique responses, 9 responses per location excluding the stimulus electrode. These 45 responses are in the velocity domain and can be overlaid on top of each other. The maximum across all 45 responses is calculated to obtain a signal that shows the maximal responses, the maximum velocity projection. Different metrics can be extracted from this by taking the area under the curve of different velocity regions and comparing the MVP's across different current values.

FIG. 14 illustrates a computer system 1400 adapted according to certain embodiments of the test controller according to the present invention. The central processing unit (“CPU”) 1402 is coupled to the system bus 1404. The CPU 1402 may be a general-purpose CPU or microprocessor, graphics processing unit (“GPU”), and/or microcontroller. The present embodiments are not restricted by the architecture of the CPU 1402 so long as the CPU 1402, whether directly or indirectly, supports the operations as described herein. The CPU 1402 may execute the various logical instructions according to the present embodiments.

The computer system 1400 also may include random access memory (RAM) 1408, which may be synchronous RAM (SRAM), dynamic RAM (DRAM), synchronous dynamic RAM (SDRAM), or the like. The computer system 1400 may utilize RAM 1408 to store the various data structures used by a software application. The computer system 1400 may also include read-only memory (ROM) 1406 which may be PROM, EPROM, EEPROM, optical storage, or the like. The ROM may store configuration information for booting the computer system 1400. The RAM 1408 and the ROM 1406 hold user and system data, and both the RAM 1408 and the ROM 1406 may be randomly accessed.

The computer system 1400 may also include an input/output (I/O) adapter 1410, a communications adapter 1414, a user interface adapter 1416, and a display adapter 1422. The I/O adapter 1410 and/or the user interface adapter 1416 may, in certain embodiments, enable a user to interact with the computer system 1400. In a further embodiment, the display adapter 1422 may display a graphical user interface (GUI) associated with a software or web-based application on a display device 1424, such as a monitor or touch screen.

The I/O adapter 1410 may couple one or more storage devices 1412, such as one or more of a hard drive, a solid-state storage device, a flash drive, a compact disc (CD) drive, a flash drive, and a tape drive, to the computer system 1400. According to one embodiment, the data storage 1412 may be a separate server coupled to the computer system 1400 through a network connection to the I/O adapter 1410. The communications adapter 1414 may be adapted to couple the computer system 1400 to a network, which may be one or more of a LAN, WAN, and/or the Internet. The communications adapter 1414 may also be adapted to couple the computer system 1400 to other networks such as a global positioning system (GPS) or a Bluetooth network. The user interface adapter 1416 couples user input devices, such as a keyboard 1420, a pointing device 1418, and/or a touch screen (not shown) to the computer system 1400. The keyboard 1420 may be an on-screen keyboard displayed on a touch panel. Additional devices (not shown) such as a camera, microphone, video camera, accelerometer, compass, and or gyroscope may be coupled to the user interface adapter 1416. The display adapter 1422 may be driven by the CPU 802 to control the display on the display device 1424. Any of the devices 1402-1422 may be physical and/or logical.

The applications of the present disclosure are not limited to the architecture of the computer system 1400. Rather the computer system 1400 is provided as an example of one type of computing device that may be adapted to perform the functions of a test controller 1001 and a data collector 1010. For example, any suitable processor-based device may be utilized including, without limitation, personal data assistants (PDAs), tablet computers, smartphones, computer game consoles, and multi-processor servers. Moreover, apparatus of the present disclosure may be implemented on application-specific integrated circuits (ASIC), very large-scale integrated (VLSI) circuits, state machine digital logic-based circuitry, or other circuitry.

The embodiments described herein are implemented as logical operations performed by a computer. The logical operations of these various embodiments of the present invention are implemented (1) as a sequence of computer-implemented steps or program modules running on a computing system and/or (2) as interconnected machine modules or hardware logic within the computing system. The implementation is a matter of choice dependent on the performance requirements of the computing system implementing the invention. Accordingly, the logical operations making up the embodiments of the invention described herein can be variously referred to as operations, steps, or modules. As such, persons of ordinary skill in the art may utilize any number of suitable electronic devices and similar structures capable of executing a sequence of logical operations according to the described embodiments. For example, the computer system 1400 may be virtualized for access by multiple users and/or applications.

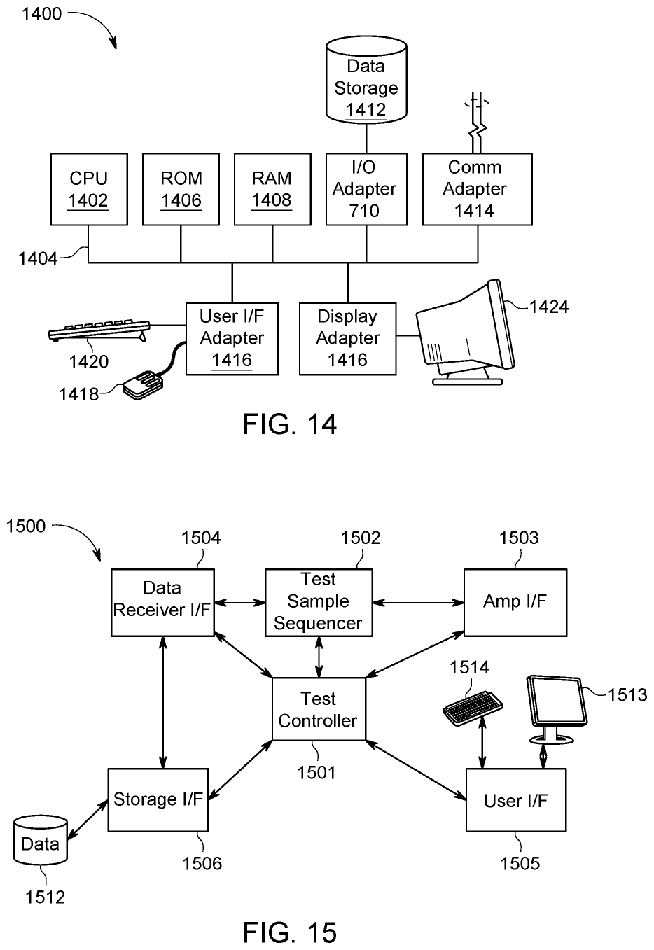

FIG. 15 illustrates a computing system of software components of an embedded electrode array controller capable of testing cell samples according to the present invention. The embedded electrode array controller 1500 includes a set of software components for activating stimulus-generating components and recording digital representations of any observed responses to the stimulus. The set of software components includes a test controller component 1501, a test sample sequencer 1502, a signal amplifier control interface 1503, a data receiver interface 1504, a user interface 1501 coupled to user display 1514 and input devices 1514, and a storage interface 1506 coupled to local memory storage devices 1512.

The test controller component 1501 receives automated test procedure commands from a user via the user interface component 1505. The test controller component 1501 also receives apparatus status indication data from the electronics within the PCB array 500 and the interface PCB 900. The test controller component 1501 interacts with the remaining set of processing components 1502-1506 to perform a sequence of operations needed to implement an automated test process as needed. The test controller component 1501 may also generate test status and test result data for the user to view on a display device 1513 via the user interface component 1505 as the operations of the automated test process proceeds

The test sample sequencer 1502 generates control signals to configure the tissue culture wells 200 within the embedded electrode array 100 to perform a test operation. The test sample sequencer 1502 with the signal amplifier control interface 1503 defines the characteristics of a stimulating signal applied to one of the electrodes 211-220 in each tissue culture well 200 of the embedded electrode array 100. The test sample sequencer 1502 may repeat this configuration for each tissue culture well 200 in use for a particular test operation. The test sample sequencer 1502 initiates the application of the stimulating signal and the recording of all responsive signals by the data collector 1010 for all tissue culture wells 200 of the embedded electrode array 100 at one time. The test sample sequencer 1502 terminates the application of the stimulating signal and the recording of all responsive signals and may reconfigure these settings and repeat the test procedure operations. The test sample sequencer 1502 may perform as many test procedure operations required under the controller of the test controller component 1501.

The test controller 1001 communicates with attached devices capable of configuring each stimulating signal to be applied to a tissue cell sample 101 within a tissue culture well 200. The signal amplifier control interface 1503 enables the test controller 1001 to change any characteristic of a stimulating signal such as amplitude, frequency, and delay between events that are assigned to each electrode 211-220 within each tissue culture well 200 of the embedded electrode array 100. The signal amplifier control interface 1503, working with the test sample sequencer 1502 may configure each tissue culture well 200 to stimulate the tissue cell sample 101 with one of its electrodes 211-220. The signal amplifier control interface 1503 performs all of the data formatting, computer-to-computer communications, encryption processing, and all similar operations needed by the test controller 1001 to communicate with the components generating stimulating signals and addressing each signal to an electrode 211-220.

The data receiver interface 1504 test controller 1001 communicates with the data collector 1010 and interface PCB 900. The data receiver interface 1504 performs all of the data formatting, computer-to-computer communications, encryption processing, and all similar operations needed by the test controller 101 to communicate with all attached devices.

The user interface 1501 coupled to user display 1514 and input devices 1514 provides input and output processing to provide a user with messages and data needed to initiate, monitor, control and terminate an automated test process. This user interface 1501 also accepts commands from the user to instruct the test controller 1001 to perform these tasks as needed. The user display 1513 may be any computer display device such as a monitor, LED flat screen, or similar device configured to display data to a user. The input devices 1514 may include pointing devices such as a mouse, trackpad, and trackball, and input devices such as a keyboard that is configured to permit a user to input data and commands into the test controller 1001.

The storage interface 1506 coupled to local memory storage devices 1512 processes all data storage operations for the test controller 1001. These operations include the writing of data into the local memory storage devices 1512, deletion of data from the local memory storage devices 1501, searching and retrieving data from the local memory storage devices 1512, and indexing the local memory storage devices 1512 to maintain efficient searching when needed.

Even though particular combinations of features are recited in the present application, these combinations are not intended to limit the disclosure of the invention. In fact, many of these features may be combined in ways not specifically recited in this application. In other words, any of the features mentioned in this application may be included in this new invention in any combination or combinations to allow the functionality required for the desired operations.

No element, act, or instruction used in the present application should be construed as critical or essential to the invention unless explicitly described as such. Further, the phrase “based on” is intended to mean “based, at least in part, on” unless explicitly stated otherwise.

What is claimed is:

1. An apparatus providing an embedded electrode array capable of testing cell samples, the apparatus comprises:
an embedded electrode array, the embedded electrode array comprises:
a plurality of tissue culture wells each having a plurality of electrode pads for containing a tissue cell sample;
one or more signal connectors having a plurality of connector pins for receiving a stimulating signal for each of the plurality of tissue culture wells and generating a plurality of responsive signals from each of the tissue cell samples; and
a plurality of circuit runs embedded within an EEA-printed circuit board, wherein each of the plurality of circuit runs connects one of the plurality of connector pins to an electrode pad within each tissue culture well;
an interface printed circuit board, the interface printed circuit board comprising:
one or more signal connectors for receiving a test signal for use as the one or more stimulating signals to a plurality of tissue culture wells in an attached embedded electrode array;
one or more interface signal connectors having a plurality of connector pins for transmitting a stimulating signal for each of the plurality of tissue culture wells in the attached embedded electrode array and receiving a plurality of responsive signals from each of the tissue cell samples; and
a plurality of amplifier-digitizer circuits, each configured to receive one or more of responsive signals organized into a plurality of data channels and to digitize the responsive signals for transmission as a data set for each of the plurality of data channels;
a test controller, the test controller configures the one or more stimulating signals and the plurality of data channels in the interface printed circuit board for receiving and sampling responsive signals into the plurality of data channels; and
a data collector, the data collector receives data sets for a plurality of data channels for processing and storage.

2. The embedded electrode array according to claim 1, wherein each of the plurality of tissue culture wells comprises:
a cell channel layer of culturing cells into the tissue cell sample;
a pair of tissue culture well positioned at each of two ends of the Cirlex channel;
8 electrode pads of the plurality of electrode pads positioned across the Cirlex channel; and
a single electrode pad of the plurality of electrode pads within each of the pair of tissue culture wells.

3. The embedded electrode array according to claim 2, wherein the embedded electrode array further comprising:
a pair of embedded copper layers within the EEA-printed circuit board for defining the plurality of circuit runs;
a base polyimide layer between the pair of embedded copper layers;
a top polyimide layer on top of one of the two copper layers; and
a bottom polyimide layer on top of one of the two copper layers.

4. The embedded electrode array according to claim 3, wherein the embedded electrode array further comprising a soft-gold electroplated layer.

5. The embedded electrode array according to claim 4, wherein each of the tissue culture wells are within the top polyimide layer exposing the plurality of electrodes within the cell channel layer and the tissue culture well.

6. The interface printed circuit board according to claim 1, wherein the test signal is received from an external function generator.

7. The interface printed circuit board according to claim 1, wherein the data sets for each of the plurality of data channels are generated by the plurality of amplifier-digitizer circuits in groups of data sets corresponding to the data set from a portion of the tissue culture wells.

8. The interface printed circuit board according to claim 7, wherein each the plurality of amplifier-digitizer circuits receives 16 of the one or more of responsive signals to generate 16 data channels.

9. The interface printed circuit board according to claim 8, wherein the interface printed circuit board comprises 16 amplifier-digitizer circuits to generated 256 data channels organized into 2 groups of 128 data channels.

10. An embedded electrode array comprising:
a plurality of tissue culture wells, each of the plurality of tissue culture wells having a plurality of electrode pads for electrically contacting to a tissue cell sample;
one or more signal connectors having a plurality of connector pins for receiving a stimulating signal for each of the plurality of tissue culture wells and generating a plurality of responsive signals from each of the tissue cell samples; and
a plurality of circuit runs embedded within the embedded electrode array, wherein each of the plurality of circuit runs connects one of the plurality of connector pins to an electrode pad.

11. The embedded electrode array according to claim 10, wherein each of the plurality of tissue culture wells comprises:
a cell channel layer of culturing cells into the tissue cell sample;
a pair of tissue culture well positioned at each of two ends of the cell channel layer;
9 electrode pads of the plurality of electrode pads positioned across the cell channel layer; and
a single electrode pad of the plurality of electrode pads within each of the pair of tissue culture wells.

12. The embedded electrode array according to claim 11, wherein the embedded electrode array further comprising:
a pair of embedded copper layers within the EEA-printed circuit board for defining the plurality of circuit runs;
a base polyimide layer between the pair of embedded copper layers;
a top polyimide layer on top of one of the two copper layers; and
a bottom polyimide layer on top of one of the two copper layers.

13. The embedded electrode array according to claim 12, wherein the embedded electrode array further comprising a soft-gold electroplated layer between the top polyimide layer and one of the two copper layers.

14. The embedded electrode array according to claim 13, wherein each of the tissue culture wells are within the top polyimide layer exposing the plurality of electrodes within the cell channel layer and the tissue culture well.

15. An interface printed circuit board, the interface printed circuit board comprising:
one or more signal connectors for receiving a test signal for use as the one or more stimulating signals to a plurality of tissue culture wells in an attached embedded electrode array;
one or more interface signal connectors having a plurality of connector pins for transmitting a stimulating signal for each of the plurality of tissue culture wells in the attached embedded electrode array and receiving a plurality of responsive signals from each of the tissue cell samples; and
a plurality of amplifier-digitizer circuits, each configured to receive one or more of responsive signals organized into a plurality of data channels and to digitize the responsive signals for transmission as a data set for each of the plurality of data channels.

16. The interface printed circuit board according to claim 15, wherein the test signal is received from an external function generator.

17. The interface printed circuit board according to claim 15, wherein the data sets for each of the plurality of data channels are generated by the plurality of amplifier-digitizer circuits in groups of data sets corresponding to the data set from a portion of the tissue culture wells.

18. The interface printed circuit board according to claim 17, wherein each the plurality of amplifier-digitizer circuits receives 16 of the one or more of responsive signals to generate 16 data channels.

19. The interface printed circuit board according to claim 18, wherein the interface printed circuit board comprises 16 amplifier-digitizer circuits to generated 256 data channels organized into 2 groups of 128 data channels.

20. The interface printed circuit board according to claim 18, wherein the interface printed circuit board further comprises a digital switch connecting 128 data channels of 1 of the 2 groups to an external data collector at a time.
